(19) United States
(12) Patent Application Publication
(10) Pub. No.: US 2026/0069184 A1
(43) Pub. Date: Mar. 12, 2026

(54) Device for Detecting Magnetic Signals Generated by a Beating Heart

(71) Applicant: Robert Bosch GmbH, Stuttgart (DE)

(72) Inventors: Florian Dolde, Stuttgart (DE); Frederik Schaal, Reutlingen (DE); Florian Krist, Stuttgart (DE); Riccardo Cipolletti, Geislingen An Der Steige (DE); Eckhard Wehrse, Stuttgart (DE)

(21) Appl. No.: 19/107,150

(22) PCT Filed: Aug. 18, 2023
(86) PCT No.: PCT/EP2023/072842

§ 371 Date: Feb. 27, 2025

(30) Foreign Application Priority Data
Sep. 9, 2022 (DE) 10 2022 209 426.3

(51) Int. Cl. A61B 5/243; A61B 5/00

(52) U.S. Cl. CPC A61B 5/243; A61B 5/6892; A61B 5/7475; A61B 2562/0223; A61B 2562/0247; A61B 2562/0271

(57) ABSTRACT

A device is for detecting magnetic signals produced by a beating heart. The device includes at least one nitrogen vacancy center, NV, a magnetometer unit, configured to sense a magnetic field strength and/or field direction, at least one further sensor, and a signal processing unit, to which the at least one NV magnetometer unit and the at least one further sensor are connected. The device is configured to, using the signal processing unit, determine at least one effective magnetic field strength and/or at least one effective field direction from the signals of the at least one NV magnetometer unit and the at least one further

The present invention relates to a device for sensing magnetic signals generated by a beating heart

### BACKGROUND OF THE INVENTION

Optically pumped or diamond-based quantum sensors based on NV centers are particularly suitable as sensors for measuring very small magnetic field strengths. DE 10 2022 204 526.2 describes a magnetometer that utilizes optically pumped and optically detected magnetic resonance (ODMR). It is thereby exploited that, under the influence of an external magnetic field, the energy levels of certain spin states of unpaired electrons are split, the so-called Zeeman effect. The splitting of the energy levels results in changed transitions during relaxation from excited states, which can then be measured, for example, by optical excitation and frequency-dependent detection of the resulting fluorescence radiation or by observation of optical properties such as the absorption of light. From the measured optical parameters, the magnetic field strength may then in turn be inferred.

### DISCLOSURE OF THE INVENTION

According to the invention, a device for sensing magnetic signals generated by a beating heart is proposed with the features of claim 1. Advantageous embodiments are the subject matter of the dependent claims and the following description.

A magnetocardiogram (abbreviated MCG) is the recording and representation of the magnetic field of the heart that arises from the electrophysiological activity of the myocardial cells. In the context of the invention, a contactless, passive approach for long-term monitoring of the human heart with high resolution is presented. This is determined by at least one nitrogen vacancy magnetometer unit (so-called NV magnetometer unit).

Specifically, a device for sensing magnetic signals generated by a beating heart is proposed, comprising at least one NV magnetometer unit which is configured to sense a magnetic field strength and field direction, at least one further sensor, and a signal processing unit to which the at least one NV magnetometer unit and the at least one further sensor are connected, wherein the device is configured to use the signal processing unit to determine at least one effective magnetic field strength and/or at least one effective field direction from the signals of the at least one NV magnetometer unit and the at least one further sensor. Both a wireless and wired connection are provided between the sensors and the signal processing unit. Such a device may also be referred to as a magnetocardiograph.

The invention relates to signal processing for magnetocardiographs, in particular but not exclusively related to applications in household environments. In this case, signal processing has the task of making it independent from external magnetic field interference variables. Wherever electrical charges move or where magnetic or magnetizable materials are moved, the ambient magnetic field changes, which must be separated from the signal by corresponding signal processing in order to make it measurable. Particularly good signal processing also enables the improvement of signal resolution and thus the detection of rarer and more difficult to detect artifacts over time in the heart signal or other biomedical signals and therefore the detection associated diseases. In this application, in particular, the advantages of coupling with other sensors and technologies will be discussed. The at least one further sensor may be advantageously used for signal processing, in particular to trigger averaging, referencing and comparison algorithms.

A particular advantage of the NV sensors is their size, specifically of the sensor medium. For the application, the active measurement volume should be small compared to the object (heart) to be measured, otherwise integration will occur over large portions of the signal due to the surface coverage, and thus the signal may disappear because the integral is zero. The smaller the active measurement volume is compared to the heart, the better the signal detection will be. NV sensor technology has a very small active sensor volume. This capability for creating small designs also allows the sensors to be used in a geometric arrangement. In particular, very high resolution arrangements are possible due to the very small active sensor volume. This also allows for ease of integration into parts of buildings (walls, ceilings, etc.) or interior objects (furniture, etc.), textiles (garments, ceilings, etc.) or other everyday objects, wherein there are numerous potential options.

Diamond NV magnetometers are based on reading out magnetic resonances from specific defect centers in diamond, in particular nitrogen vacancies (NV), which occur as impurities in the carbon lattice of diamond and can also be introduced in a targeted manner. If the NV center is optically excited in the ground state, e.g., by radiating in a pump laser beam having a suitable wavelength (in this case in the green wavelength range, e.g., at 532 nm for off-resonance excitation), the electrons are lifted from the triplet ground state to the excited triplet state and relax while emitting fluorescence light in the red wavelength range at 650-800 nm (637nm=zero phonon line). Since the probability of non-spin-conserving transitions from the spin state is higher when the spin quantum number mₛ=±1, continuous excitation pumping ensures that the NV centers are largely hyperpolarized in the spin state mₛ=0.

There is an energy difference between the mₛ=0 and mₛ=±1 spin states in the ground state, which in this case is about 2.87 GHz. Thus, if microwave radiation is radiated into the diamond in addition to the optical excitation, there is a dip in the red fluorescence at this resonance frequency of 2.87 GHz, because the spin-polarized electrons are lifted by the microwave field from mₛ=0 to the mₛ=±1 ground state and excited from there by the pump light to the mₛ=±1 excited state. From there, however, primarily non-radiative transitions and weak infrared fluorescence transitions occur via the singlet state, and the fluorescence in the red range ceases.

If an external magnetic field is present, the so-called Zeeman effect leads to the splitting of the otherwise energetically equal mₛ=±1 triplet levels into energetically equidistant Zeeman levels. Plotting the fluorescence against a frequency spectrum of the microwave excitation then reveals two dips in the fluorescence spectrum, the frequency spacing of which is proportional to the magnetic field strength of the external magnetic field. The magnetic field sensitivity is primarily defined by the minimally resolvable frequency shift and can reach up to 1 pT/√Hz or less. Because the nitrogen vacancy center in the single-crystal diamonds has four possible ways to position itself in the crystal lattice, the presence of a directed magnetic field causes the nitrogen vacancy centers present in the crystal to react differently to the external magnetic field depending on their position in the crystal. As a result, four pairs of fluorescence minima may appear in the spectrum, from the shape and position of which both the magnetic field strength as an amount and the direction of the external magnetic field are unambiguously determinable.

In order to enable vectorial magnetic field measurements, the device in one embodiment comprises a device for generating a substantially homogeneous bias magnetic field in the area of the magnetometer units or their sensor media, wherein the bias magnetic fields of different NV magnetometer units are expediently different. This may be a Helmholtz coil assembly, wherein at least the sensor medium is disposed within the Helmholtz coil assembly (each magnetometer unit more expediently has its own bias field, which improves field determination). It can also be other equipment such as a simple coil, a long coil, permanent magnet solutions such as in a Hallbach array, etc.

Cardiac signals have a magnetic signature at a distance of some cm with an amplitude of (only) picotesla (pT), whereas, for example, the Earth's magnetic field in central Europe is approximately 50 μT (microtesla), i.e., it is more powerful by a factor of 10⁶. However, even such small field strengths are resolvable with a high level of precision for a long period of time with the proposed technology. For example, a magnetic shield or a gradiometer circuit may be used for this purpose.

High resolution detection of the accurate heart signal can be used to detect a variety of diseases such as permanent atrial fibrillation and paroxysmal atrial fibrillation. Thus, a heart attack and subsequently a stroke (particularly after an unrecognized heart attack) can be prevented. Further, the invention is suitable for early detection of an ST segment elevation infarction, a different kind of elevation infarction, a pulmonary embolism, an AV nodal reentry tachycardia, ventricular extrasystoles, as well as very rare pathogenic diseases such as arrhythmogenic right ventricular tachycardia, which can otherwise only be detected by gene sequencing.

Only an accurate resolution of the heart signal allows for this disease detection. Namely, in the case of very noisy or poorly resolved signals, the displacements of the various PQRST complexes of the heart compared to each other or over time, fluctuations in their amplitude, deformations or minor disorders are not detectable. However, this resolution of these criteria are important factors because the abovementioned problems can lead to mixing up complexes (e.g. interpreting an elevated and delayed T-wave as an R-wave, which is however common in a “healthy” heart), and to false alarms.

When a gradiometer circuit of at least two NV magnetometer units is used, and therefore a geometric arrangement of at least two NV magnetometer units, one magnetometer unit is always a greater distance from the heart (as a relatively weak magnetic field source) than another magnetometer unit. Due to the gradiometer circuit, i.e. substantially (vectorial) subtraction of what is measured, the magnetic field gradient approximately corresponds to the field originating from the weak source, while substantially stronger background fields (which are substantially the same in both magnetometer units) are eliminated. This eliminates the need for magnetic shielding so that magnetic field measurement can be carried out in everyday environments. Accordingly, the invention is particularly suitable for non-shielded measurement of weak magnetic fields. Technical details for gradiometer solutions that can also be used in the context of the present invention are disclosed in DE 102022201690.4 and are to be incorporated herein.

In one embodiment, the device is configured to detect a magnetic field strength and field direction by means of the at least one NV magnetometer unit. Another advantage of the NV sensor technology is the direction or vector information. In contrast to other technologies, this is intrinsic in NV sensor technology. Thus, interferences do not need to be introduced through modulation techniques and/or less favorable projections do not need to be used, nor do multiple separate sensors need to be used. The vector and gradiometry information is thus available at the exact same location (diamond size, i.e. single digit mm\hat{ }3 and below) and not separated by a few cm to many cm as with other technologies. With NV magnetometer units, which can determine not only the field strength but also the direction of the magnetic field, improved suppression of a background field and thus better detection of signals with superimposition of strong interference signals are enabled.

In one embodiment, the device is configured to determine a repeating reference point in the signals of the at least one NV magnetometer unit by means of the at least one further sensor, to divide the signals of the at least one NV magnetometer unit into individual signal sections based on the reference point and to determine an effective signal section from the signal sections. Thus, in particular, averaging may occur over multiple heartbeats. To resolve heart signals with high accuracy, multiple heartbeats can be averaged to reduce noise. Since a typical heartbeat is slightly irregular, triggering by a further sensor, e.g. by a pressure sensor or a pulse oximeter, is proposed for this purpose. In this case, short averaging (e.g., over 5-10 cycles) and long averaging (e.g., over 100-200 cycles) are possible in order to obtain different information.

Also, in particular, it is possible to consider the original signals of the at least one NV magnetometer unit, of the at least one further sensor and/or of the effective signal section together for the purpose of analysis. The at least one further sensor may also provide a comparative signal or additional signal for directly distinguishing artifacts. Further, the at least one further sensor may also be used to identify additional peaks, e.g. to distinguish an elevated T-wave from an R-peak. In particular, the NV magnetometer unit can precisely resolve the signal curve so that the individual waves are clearly identifiable, and the at least one further sensor can detect, for example, strong spikes. If there is an additional “strong spike,” then a reference sensor would simply assume a somewhat higher pulse, which would not be abnormal. By comparing the signals of the NV magnetometer unit and those of the additional sensor, it is possible to detect, e.g., a tall T-wave (indicator of a heart attack). Furthermore, signals in the magnetic signature are in some cases different than differently detected peaks, in particular in different measurement channels (vectometry). Here too, additional features can very easily be detected by a corresponding comparison. Furthermore, if averaging is carried out to an external trigger, which does not detect an additional peak, then the additional peak can be identified as a clear signal increase of a specific wave, even with long averaging times, because the peak only occurs in one of the signals.

In practice, in devices with arrangements of at least two NV magnetometer units, one NV magnetometer unit or one (gradiometer) arrangement of two NV magnetometer units is typically always particularly close to the heart. Their signal can be advantageously used in particular, since it is to be assumed that it best represents the magnetic signal of the heart. This signal may be offset with others to form, for example, vectorial derivatives (tensiometry). It can also be used to determine which other NV magnetometer units are very far from the heart and therefore are better used for determining the background. In this case, the position of the user, in particular also a position that changes over time, can be determined by the at least one further sensor, e.g. pressure or force sensors (or by the magnetic signal itself) to thus adjust the timing of the signal processing to the changed position.

Sensing the breathing (and similar signals) via the at least one further sensor (e.g. microphone or pressure sensor) also makes it possible to correct interfering factors, since breathing means movement, which corresponds to a movement of the body relative to the NV magnetometer units. Additional information, such as the progression of breathing, may also thus be determined to infer sleep apnea or the general state of health.

In particular, a calculation specification can be determined by means of the at least one further sensor, according to which the at least one effective magnetic field strength and/or at least one effective field direction is determined from the signals of the at least one NV magnetometer unit. In particular, such a calculation specification may indicate the height at which the signals of a certain NV magnetometer unit are included the result, as well as which NV magnetometer unit this is. For example, the position of the user as described above can be a temperature, e.g. of individual NV magnetometer units, a strength or variance of a background or interference field, etc.

The resulting “time-changing” signal processing is also helpful for assessing the data and qualifying which data is to be evaluated—and which are assessed as “faulty,” for example because the user has moved too often and/or too quickly, or the like.

Temperature monitoring can also help increase safety, e.g. to induce an emergency shutdown.

Further external influences, such as temperature, pressure, the heat of another person in bed, etc., can also be detected by the at least one further sensor and thus corrected or additionally used.

A recommendation can also be derived from the data obtained, e.g. “place the pad a little lower” or “another pillow would be healthier for you” and output via an interface, e.g. on an app. In particular, this can also be coupled to other technologies, such as a smart watch and the sensors integrated in the watch, as the at least one further sensor.

The at least one further sensor may be selected from the group comprising pressure sensor, force sensor, temperature sensor, accelerometer, inertial sensor (IMU), gyroscope, pulse oximeter, microphone, magnetometer unit without nitrogen vacancy centers.

A sampling rate which is higher than the cardiac signal, in particular greater than 50Hz, is required for the application in order to resolve the signal. A range of 200 Hz to 400 Hz is considered to be particularly advantageous. Higher is always better for resolution, however this increases the requirements for sensitivity.

Further advantages and embodiments of the invention are shown in the description and the included drawings.

The invention is shown schematically in the drawings on the basis of exemplary embodiments and is described hereinafter with reference to the drawings.

### BRIEF DESCRIPTION OF THE DRAWINGS

FIG. 1 shows in a schematic block view the essential components of an NV-center magnetometer as may be used in the context of the invention.

FIG. 2 shows possible assemblies of NV magnetometer units of a device for detecting magnetic signals according to one embodiment in various figures a) to c), respectively in a schematic block view in each case.

FIG. 3 shows a diagram of a user and a device according to one embodiment of the invention in a side view.

FIG. 4 shows a diagram of possible configurations of assemblies with one or more NV magnetometer units according to embodiments of the invention in three top views a) to c).

FIG. 5 shows a diagram of possible configurations of devices with multiple assemblies with NV magnetometer units and a signal processing unit according to embodiments of the invention in two side views a) and b).

### EMBODIMENTS(S) OF THE INVENTION

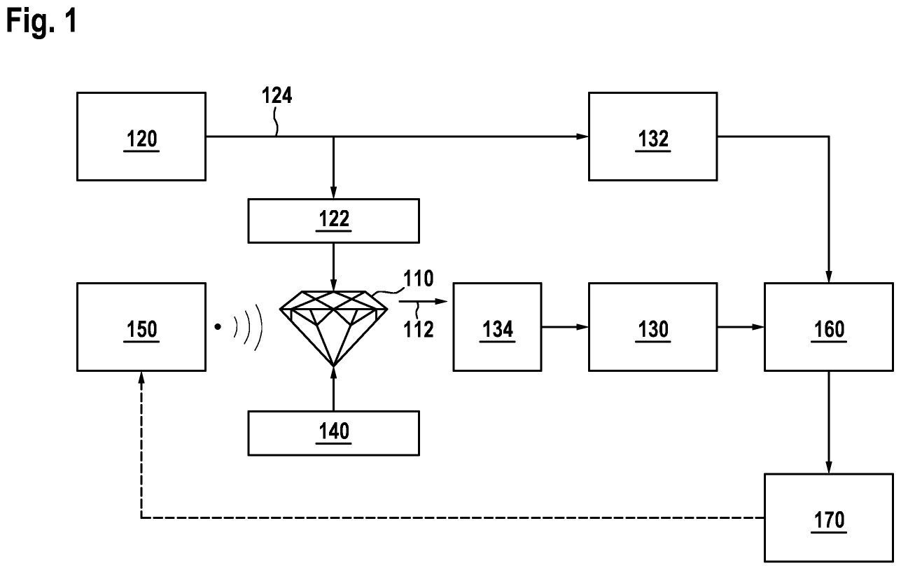

FIG. 1 shows a diagram of the essential components of an NV-center magnetometer. First, a diamond 110 with nitrogen vacancies (NV) is present as the sensor medium. The optical excitation of the NV centers is achieved by a suitable light source 120, such as an LED or a pump laser. Here, for example, a frequency-doubled Nd:YAG laser or semiconductor laser in the green range of about 510-532 nm, e.g. at 532 nm, is suitable for off-resonance excitation. Alternatively, LEDs in suitable wavelength ranges may also be used. Depending on the assembly, the light of the light source 120 may be radiated via suitable optical elements 122 such as mirrors, beam splitters, focusing optics such as lenses, and optionally via fiber optic elements in the diamonds 110. In addition, the excitation light may be irradiated continuously or in a pulsed manner by the laser such that, for example, time windows for interference-free fluorescence light measurement are kept clear.

Furthermore, a microwave source 150 may be present in the magnetometer capable of generating an electromagnetic field across a bandwidth that sufficiently covers the desired resonance frequency in the sensor medium, e.g., in the range of the NV centers in the diamonds 110. A microwave resonator structure may be used to homogeneously distribute the generated microwaves across the volume of the sensing range in the diamond. The resonator structure or microwave source 150 is preferably tuned for the frequency of the electron spin resonances. To enable vector magnetometry, an additional static bias magnetic field 140 is generated. As a result, the measurement becomes intrinsically vectorial. Different spatial directions are used in the crystal structure for this purpose. For example, to generate such a magnetic field 140, a Helmholtz coil is suitable, in which a substantially homogeneous magnetic field can be generated in a limited range by means of a pair of coils.

The resulting fluorescent light 112 from the diamond 110 may in turn be directed via suitable optical elements 134 such as optical filters, beam splitters, lenses, and/or fiber optic elements to a first photodetector 130 that is sensitive at least in the range of the fluorescence wavelength. The first photodetector 130 may also be disposed directly on the diamond 110. A second photodetector 132 is arranged to detect at least a portion of the excitation light of the light source 120, which may be decoupled by, for example, a beam splitter, a filter, or a partially permeable element. This excitation light detector signal 132 may be used as a reference signal to eliminate background signals, for example, by modulating the excitation light by way of a lock-in amplifier, and highlighting the resonance signal of interest. Additionally, or alternatively, this reference signal may be used to account for fluctuations in excitation light. Corresponding circuitry 160, such as a pre-amplifier, log amplifier, lock-in amplifier, signal filters, or others, is thus provided to receive the signals from the first and second photodetector and to pre-process the signals in an appropriate manner for further evaluation. Finally, the pre-processed fluorescence signal may be evaluated by a signal processing unit 170, e.g., with a suitable microcontroller or processor, to obtain the desired parameters of the detected magnetic field from the signal, in particular the magnetic field strength and the direction of the magnetic field.

Of course, such a device may also comprise further units not shown, such as communication units or interfaces to output measurement results. Such a device may also be advantageously integrated into an ASIC or FPGA.

In order to be usable in an everyday environment, magnetic fields not originating from desired weak sources are to be eliminated as far as possible from the measurement, in particular the Earth's magnetic field in the range of 10⁻⁵ Tesla (several microtesla). In contrast, cardiac magnetic fields are in the range of 10⁻¹² Tesla (picotesla).

The elimination of the background magnetic fields may be achieved by a shield or a gradiometer assembly for the magnetic field measurement, according to exemplary embodiments. Gradiometers are generally referred to as sensor units that are capable of detecting not only the field strength, but also the gradient of the field.

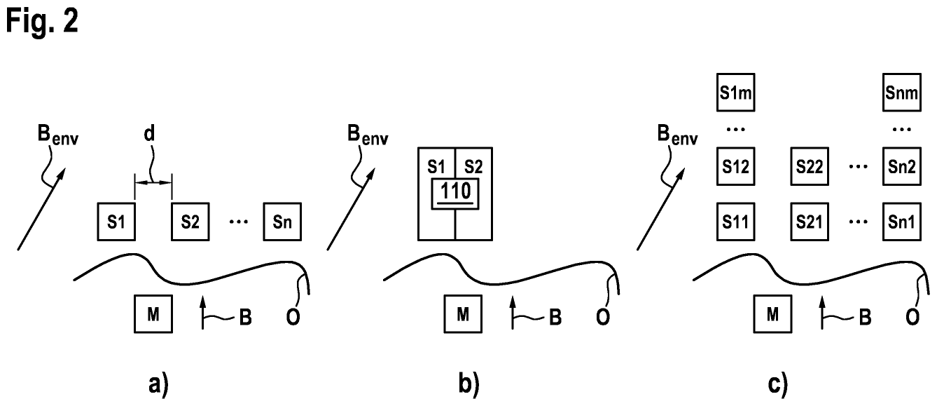

At least two individual magnetometers may be used for this purpose which are arranged at different spatial locations. As an example, a sensor unit that uses two or more NV center magnetometers in a gradiometer assembly is described below in conjunction with FIG. 2.

FIG. 2 shows possible geometric assemblies of NV magnetometer units of a device for sensing magnetic signals according to one embodiment in various figures a) to c). Figure a) shows a side view of an assembly of NV magnetometer units S1, S2, . . . Sn in any arrangement with respect to one another in a plane (perpendicular to the drawing plane, i.e. only the first row is visible). Figure b) shows a side view of two NV magnetometer units S1, S2 whose sensor media are a section of the same diamond crystal 110. Figure c) shows a side view of a number (n by m) of NV magnetometer units S11, S21, . . . Sn1, S12, S22, . . . Sn2, S1m, . . . Snm in any three-dimensional arrangement. Further layers join behind the drawing plane so that, overall, a type of cubic grid is formed. At least one NV magnetometer unit (not shown) lying in one of the rear layers, for example, is not disposed in the same plane (drawing plane) in which other NV magnetometer units S11, S21,. Sn1, S12, S22, . . . Sn2, S1m, . . . Snm are disposed.

Furthermore, M designates a signal source, here a heart, and O designates an optional surface (especially the skin of the body), which limits the accessibility to and of the magnetic field source M.

In embodiments of the inventions, more than two (but at least two) NV magnetometer units in total may form one gradiometer. With each additional NV magnetometer unit, the background field can be determined better and the location and strength of the exciter can be better separated from the background.

In other embodiments of the invention, two NV magnetometer units may also always form a gradiometer, wherein multiple gradiometers are then formed overall—depending on the number of NV magnetometer units—and the signal of interest is detected. An effective measurement signal can then be formed therefrom, in particular from the signal processing unit, for example by averaging, summation, etc.

A distance d between two NV magnetometer units S1, S2 . . . or more precisely their sensor media, corresponds to the distance between the locations where magnetic field measurements are taken simultaneously. As long as the distance between the measurement locations is relatively small, it can be assumed that the strength of an additional background magnetic field Beny is approximately equal at both locations. In contrast, the weak magnetic field B of interest will decrease significantly with increasing distance from the magnetic field source M.

Thus, by disposing two NV magnetometer units at different distances and angles from the source or from the heart, the background field can be eliminated or determined by vector arithmetic, thereby determining the small magnetic field of interest and characterizing its source (location and orientation). This may be further improved by a remote magnetometer that is far enough away that the weak magnetic field of interest has dropped below the detection threshold. With such a configuration, local changes in the background field can be compensated for by the at least two near magnetometers. For this purpose, for example, two NV magnetometer units may be arranged one above the other in an axial gradiometer configuration, such that one NV magnetometer unit of a first layer forms a gradiometer with an NV magnetometer unit located below it in a second layer below.

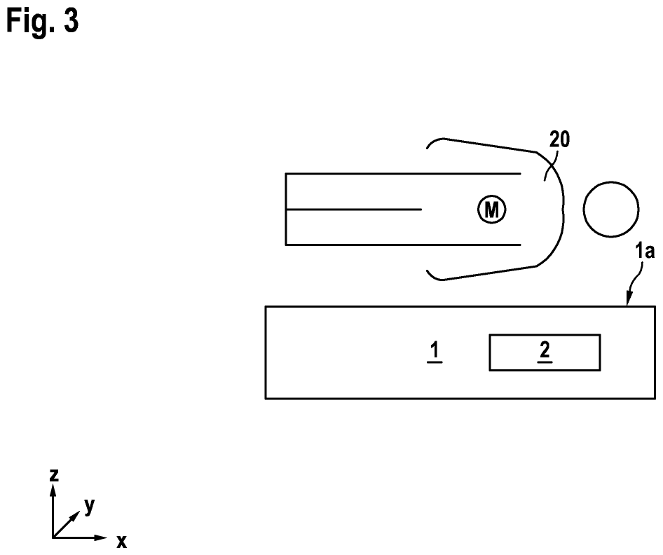

Diagrams of possible embodiments of the invention are shown in FIGS. 3 to 5, and will be described in more detail below. Like elements are provided with like reference numerals and are not described multiple times.

A device 2 is shown in each case for sensing magnetic signals, which in the examples shown comprises a body, such as a support body 1 with a support surface 1a, and at least one assembly 3 of at least one NV magnetometer unit 4, wherein the at least one assembly 3 is embedded in the body. The device 2 is used to capture magnetic signals generated by a beating heart (M), but can generally capture all magnetic signals, in particular bio-signals, i.e. those emanating from living beings.

For the purpose of illustration, the figures each have a coordinate system at the top left, wherein the drawing plane shows the x-z plane and the y-axis extends into the drawing plane.

The body here is a support body 1 adapted to receive a user 20 seated or lying on the support surface. However, it may also be a part of a building, a furnishing or textile, etc. In FIG. 3, a mattress is shown as a support body 1, as it can also be used for long-term monitoring, in particular of cardiac magnetic signals.

A device comprises an assembly or more than one assembly. The assemblies may also be arranged in a particular geometric arrangement, for example arranged in a line (1D), a plane (2D) or distributed in space (3D). As explained, two NV magnetometer units can always form a gradiometer, wherein—depending on the number of NV magnetometer units—a total of several gradiometers are formed and the signal of interest is detected. An effective measurement signal can then be formed therefrom, in particular from the signal processing unit, for example by averaging, summation, etc.

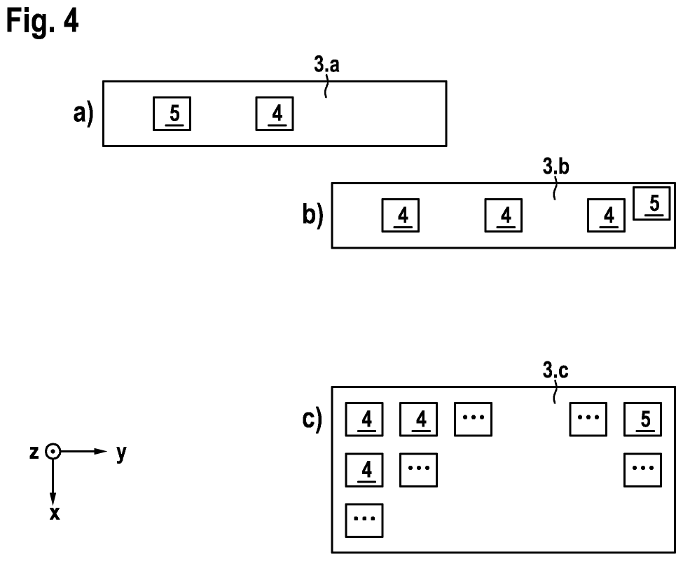

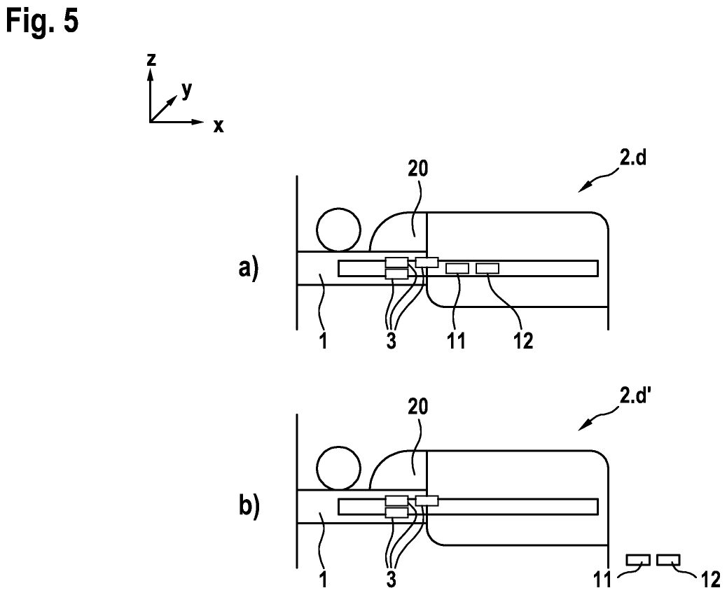

FIG. 4 shows a top view of a diagram in different views a) to c) of variants 3.a to 3.c of assemblies 3 with one or more NV magnetometer units 4, each with one or more further sensors 5. The sensors 5 may in particular be pressure sensors, pulse oximeters, temperature sensors, electrodes, etc. The NV magnetometer units 4 and/or the sensors 5 of an assembly 3 can be arranged in a particular geometric arrangement, for example in a line (1D), plane (2D) or distributed in space (3D), as previously explained in connection with FIG. 2 or 5.

In FIG. 5, different variants 2.d, 2.d′ of a device 2 with two assemblies 3 in the area of a top side and one assembly 3 in the area of a bottom side of a support body 1 are shown in two side views a) and b). Further, the assembly comprises a signal processing unit 11 to which the NV magnetometer units of the assemblies 3 are connected to determine one or more effective magnetic field strengths and/or field directions. Further, a communication unit 12 may be provided to connect the device 2 to other devices such as a PC, tablet PC, smartphone for input and output, as well as operation. The communication unit 12 may comprise, for example, wired and/or wireless interfaces. In this case, in variant 2.d, the signal processing unit 11 and communication unit 12 are also integrated in the support body and in variant 2.d′ they ae arranged outside of the support body.

Preferably, the communication unit 12 comprises an interface for communicating results, e.g., to a cardiologist or relative, or to the person using them.

An interface may include, for example, a WiFi interface or a wireless interface for providing Internet access, for example. Internet access is expediently provided with up-to-date security standards in terms of encryption, authentication, access restriction, etc. Coupling to a terminal via Bluetooth, for instance, e.g. in connection with an app, is also conceivable here, in order to indirectly establish access to the Internet, for example.

In one exemplary embodiment, an update (software renewal) and/or upgrade (software extension) can also be carried out via the interface. For example, software may be provided for download for this purpose. The device then comprises corresponding hard disk memory as well as corresponding software for updating or supplementing software modules. Advantageously, software modules for specific diseases or software modules for specific functions are offered. Software modules may each be individually classified as a medical device, if necessary.

In one exemplary embodiment, the interface may comprise an interface for (regularly) querying licensing data, e.g., for the mentioned software modules, as well as for deactivation after a license expires.

In one exemplary embodiment, the interface may also be used to make emergency calls, i.e., may represent or comprise an emergency call interface. In acute cases, an emergency call is placed automatically. In addition to the emergency call interface, the interface may also comprise a backup emergency call interface.

In one exemplary embodiment, the interface may comprise a direct interface to a communication center, for example, directly to a physician (cardiologist) or physician assistant for transmitting abnormal data sets. For telemedicine in particular, it is important that certain data sets are transmitted to a cardiologist for review via a secure interface.

In one exemplary embodiment, the interface may comprise an interface to a cloud. Cloud access should be integrated to process data sets or also to learn from data sets (machine learning on collected data sets). It may be important to introduce a step for anonymizing the data. Here as well, a safety concept is beneficial.

In one exemplary embodiment, the signal processing unit may comprise an analysis unit. In a conventional use of the device, e.g. as a long-term MCG, a large quantity of data can be generated that would be very difficult to review manually. Therefore, it is advantageous for selected abnormal data (i.e. data with certain characteristics) to be transmitted via a pre-analysis. Of course, such an analysis unit can also be provided externally to the recipient, i.e. first all data is transmitted and then the conspicuous data are selected by the recipient.

In one exemplary embodiment, the interface may be an interface to a mobile platform such as an app (app of the user, app of several users, e.g., a relative). In particular, the user, as well as relatives, can then access selected data, selected notes, and a selected user interface via a mobile platform. This is also important for relatives or, for instance, nurses for monitoring. The application may also be available for booking as an add-on for the technology. In this case as well, a security concept and an internet interface are advantageous. This can also create a licensing option that enables the review of a subscription model.

1. A device for sensing magnetic signals generated by a beating heart, the device comprising:
at least one nitrogen vacancy center, NV, magnetometer unit configured to sense a magnetic field strength and/or field direction;
at least one further sensor; and,
a signal processing unit to which the at least one NV magnetometer unit and the at least one further sensor are connected,
wherein the device is configured to determine at least one effective magnetic field strength and/or at least one effective field direction, using the signal processing unit, from the signals of the at least one NV magnetometer unit and the at least one further sensor

2. The device according to claim 1, wherein the device is configured to determine a repeating reference point in the signals of the at least one NV magnetometer unit, to divide the signals of the at least one NV magnetometer unit into individual signal sections based on the reference point, and to determine an effective signal section from the signal sections using the at least one further sensor.

3. The device according to claim 1, wherein the device is configured to determine a position of a user using the at least one further sensor, and to determine from the position of one of the at least one NV magnetometer unit whether it is close to the heart or far away from the heart.

4. The device according to claim 1, wherein the device is configured to determine a calculation specification using of the at least one further sensor, and according to the calculation specification, to determine the at least one effective magnetic field strength and/or at least one effective field direction from the signals of the at least one NV magnetometer unit.

5. The device according to claim 1, wherein the at least one further sensor is configured to detect at least one measured variable selected from a heartbeat, an electric field, an oxygen saturation in blood, a pressure, a force, sound, a temperature, an acceleration, a magnetic field strength, a magnetic field direction.

6. The device according to claim 1, wherein the at least one further sensor is a magnetometer unit without nitrogen vacancy centers.

7. The device according to claim 1, wherein:
the at least one NV magnetometer unit as a sensor medium comprises a diamond crystal or a portion of a diamond crystal with nitrogen vacancy centers, and
the device is configured to sense a magnetic field strength and/or field direction by reading out a spin resonance dependent on the magnetic field strength in the sensor medium.

8. The device according to claim 7, further comprising:
at least one excitation light source configured to radiate light into the sensor medium;
at least one microwave source configured to generate generating a resonant field in the sensor medium; and
at least one photodetector configured to sense resonance-dependent fluorescent light from the sensor medium.

9. The device according to claim 8, wherein at least two NV magnetometer units are associated with a same excitation light source and/or a same microwave source.

10. The device according to claim 7, wherein the sensor medium of at least two NV magnetometer units each comprises a section of a same diamond crystal.

11. The device according to claim 7, wherein a distance between a sensor media of at least two NV magnetometer units is between 1 and 30 millimeters.

12. The device according to claim 1, further comprising a data interface.

13. The device according to claim 12, wherein the data interface comprises at least one of WiFi interface, a cellular interface, an Internet interface, a direct interface to a communication center, an interface for querying licensing data, an interface to a cloud, and an interface to a mobile platform.

14. The device according to of claim 12, wherein the device is configured to enable a software update and/or software upgrade using the data interface and/or to make an emergency call.

15. The device according to claim 1, further comprising an analysis unit configured to select data having certain properties from the magnetic signals.
